(19) United States
(12) Patent Application Publication
(10) Pub. No.: US 2021/0007888 A1
(43) Pub. Date: Jan. 14, 2021

(54) WEARABLE REHABILITATION AND RELAXATION AID

(71) Applicant: Jarviel Rayford, Sr., Sacramento, CA (US)

(72) Inventor: Jarviel Rayford, Sr., Sacramento, CA (US)

(21) Appl. No.: 17/036,794

(22) Filed: Sep. 29, 2020

Related U.S. Application Data
(63) Continuation-in-part of application No. 15/679,012, filed on Oct. 20, 2017.
(60) Provisional application No. 62/512,077, filed on May 29, 2017.

(51) Int. Cl. A61F 7/08; A41D 13/005; A61F 13/10; A61F 13/14; A61F 13/06; A61F 13/08

(52) U.S. Cl. CPC A61F 7/086; A41D 13/0058; A61F 13/107; A61F 2007/0032; A61F 13/146; A61F 13/064; A61F 13/08; A61F 13/10

(57) ABSTRACT

A wearable rehabilitation and relaxation sleeve aid is described that provides support and therapeutic benefits to an injured area. The wearable sleeve aid includes a first inner protective layer and an outer protective layer that form a pocket area between them. A removable pack may be inserted in the pocket area. The removable pack may be cooled or heated to bring the removable pack to the desired temperature. The wearable sleeve aid further provides compression to the targeted recovery area of a user's body to reduce swelling and pain.

### CROSS-REFERENCE TO RELATED APPLICATIONS

This application is a continuation in part application which claims priority to U.S. Provisional Patent Application No. 62/512,077, which was filed on May 29, 2017, and to U.S. Non-Provisional patent application Ser. No. 15/679,012, which was filed on Oct. 20, 2017, which are incorporated by reference in their entirety.

### FIELD OF THE DISCLOSURE

The present invention relates to a rehabilitation aid in the form of a wearable sleeve aid that can be used for any part of the body that requires rehabilitation or relaxation.

### BACKGROUND

The present invention relates to rehabilitation and, more particularly, to a wearable rehabilitation and relaxation aid.

Systems currently used are either limited, or assisted by other applications of bandages, wraps, tapes, or braces. Presently available systems are big and bulky, can only be used one time, and may require a user to be immobilized because the user has to stay connected to a plug or an outlet. Further, the currently available systems for rehabilitation do not provide coverage for the supportive muscles in conjunction with the injured area. A major problem with existing rehabilitation aids is that the injured area is exposed to uncomfortable outside elements, such as a very cold temperature or an extremely hot temperature of the existing rehabilitative aids, which could compound the discomfort to the user. Further, existing rehabilitative aids fall off easily and are hard to keep secured in place, meaning that the user does not get the full benefit of the rehabilitative aid and cannot walk around or move freely while wearing them. Accordingly, there is still a need for an improved wearable rehabilitation and relaxation aid.

### SUMMARY

According to one embodiment, one or more embodiments are provided below for a wearable sleeve aid configured to function as a rehabilitative and relaxation aid for a targeted recovery area of a body. In one or more non-limiting embodiments, the wearable sleeve aid may include a first inner protective layer and a second outer protective layer. The wearable sleeve aid may further include a pocket area, whereby the pocket area is located between the first inner protective layer and the second outer protective layer. The wearable sleeve aid may further include a removable pack, whereby the removable pack is configured to be inserted into the pocket area formed between the first inner protective layer and second outer protective layer. An external securing device may be attached to either the first inner protective layer or the second outer protective layer or both layers, whereby the external securing device is configured to provide additional compression as well as to secure the removable pack in place when the removable pack is positioned within the pocket area.

The present description may further include an exemplary method of using a wearable sleeve aid. The method may include selecting a wearable sleeve aid having a removable pack that may be located in a pocket area of the wearable sleeve having proper pack placement for a targeted recovery area of a user's body, whereby the proper pack placement is injury specific or site specific. The method may further include bringing the removable pack to a desired temperature. Upon reaching the desired temperature, the removable pack may be inserted into a pocket area located within the wearable sleeve aid. The method may further include positioning the wearable sleeve aid on the targeted recovery area on the user's body by sliding the wearable sleeve aid over the targeted recovery area. The method may further include providing additional compression to the targeted recovery area via an attached external securing device. This may include wrapping one or more straps over the pocket area of the wearable sleeve, whereby the one or more straps are attached to the wearable sleeve aid. Upon completion of use, the wearable sleeve aid may be removed by pulling the wearable sleeve from the user's body.

Other aspects and advantages of the invention will be apparent from the following description and the appended claims.

### BRIEF DESCRIPTION OF THE DRAWINGS

Embodiments of the present disclosure are described in detail below with reference to the following drawings. These and other features, aspects, and advantages of the present disclosure will become better understood with regard to the following description, appended claims, and accompanying drawings. The drawings described herein are for illustrative purposes only of selected embodiments and not all possible implementations and are not intended to limit the scope of the present disclosure.

FIG. 1 is a pictorial illustration of an embodiment for a wearable sleeve aid for use on an elbow and arm of a user in accordance with an illustrative embodiment.

FIG. 2 is an exploded view of one or more layers of a wearable sleeve aid in accordance with an illustrative embodiment.

FIG. 3 is a sectional view taken along line 3-3 in FIG. 1 of a wearable sleeve aid in accordance with an illustrative embodiment.

FIG. 4 is a pictorial illustration of an embodiment for a wearable sleeve aid for a user's foot and lower part of the leg in accordance with an illustrative embodiment.

FIG. 5 is a pictorial illustration of an embodiment for a wearable sleeve aid for a user's back and shoulder area in accordance with an illustrative embodiment.

FIG. 6 is a flow chart of an exemplary method of using a wearable sleeve aid in accordance with an illustrative embodiment.

### DETAILED DESCRIPTION

In the Summary above and in this Detailed Description, and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

The term “comprises” and grammatical equivalents thereof are used herein to mean that other components, ingredients, steps, among others, are optionally present. For example, an article “comprising” (or “which comprises”) components A, B, and C can consist of (i.e., contain only) components A, B, and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm and upper limit is 100 mm.

Certain terminology and derivations thereof may be used in the following description for convenience in reference only and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

The term “coupled to” as used herein may mean a direct or indirect connection via one or more components.

The present description includes one or more non-limiting embodiments for a wearable sleeve aid. In one or more non-limiting embodiments, the presently disclosed invention is configured to function as a rehabilitative and relaxation aid for a targeted area of the body. Most rehabilitative aids appear in the form of slings, wraps, or braces, such as, for example, a knee brace, arm brace, back wrap, etc. The user usually can select a rehabilitative aid for a specific body part that the user has to wrap around or slide over an injured area of the body. However, a problem for existing rehabilitative aids is that in order for the user to also apply a heating or cooling pack, the user has to separately hold the heating or cooling pack onto the rehabilitative aid or use an electrically powered heating or cooling pack that forces the user to be stationary and near enough to a plug or outlet for the heating or cooling pack to reach a desired temperature. \

Because the user has to come up with their own way to hold the heating or cooling pack on the injured part of the user's body, it often occurs that the heating or cooling pack is not located near enough to the injured area for the injured area to benefit properly from the heating or cooling pack and may not use the rehabilitative aid for a long enough period of time to benefit from the heating or cooling pack.

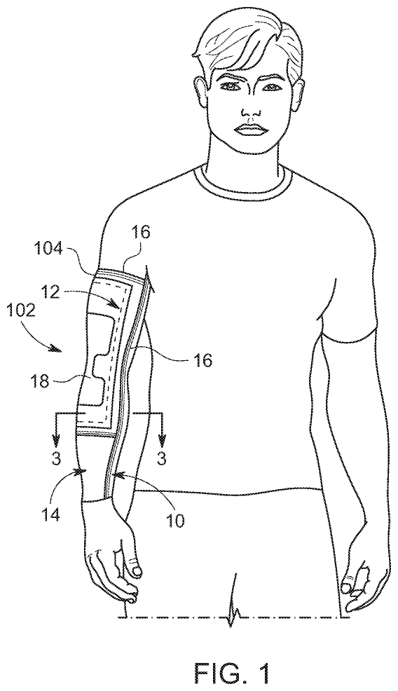

The one or more embodiments described herein for a rehabilitative aid provides a user with a wearable sleeve aid that can act as a brace and support to any part of the user's body needing a brace or support, such as, but not limited to, a user's knees, ankles, shins, calves, hands, elbows, arms, shoulders, back, neck. Accordingly, the wearable sleeve aid (e.g. wearable sleeve aid 102 as shown and as described in FIGS. 1-6) can be worn as a helpful brace or support to provide support to a targeted part of a user's body. Additionally, the wearable sleeve aid (e.g. wearable sleeve aid 102 as shown and as described in FIGS. 1-6) described herein in one or more non-limiting embodiments further includes the proper heating or cooling pack placement within the wearable sleeve aid 102 so as to provide closer contact to the targeted area of the user's body requiring rehabilitation and healing. Further details are provided with respect to the Figures.

Referring now to FIG. 1, FIG. 1 is a pictorial illustration of a wearable sleeve aid in accordance with a non-limiting embodiment. The wearable sleeve aid 102 shown in FIG. 1 may be representative of a wearable sleeve aid 102 that may be used for other parts of the body and that incorporate the described components of the wearable sleeve aid 102 shown in FIG. 1. FIG. 1 shows the wearable sleeve aid 102 configured to slide over the arm and elbow of the user. In one or more non-limiting embodiments, the wearable sleeve aid 102 has a first inner protective layer 10 and a second outer protective layer 14 that form a pocket area 104. In a non-limiting embodiment, a form of stitching 16 is used to attach the first inner protective layer 10 to the second outer protective layer 14, whereby a pocket area 104 is formed between the first inner protective layer 10 and the second outer protective layer 14. In one or more non-limiting embodiments, the stitching 16 extends around the outer boundaries of the wearable sleeve aid 102. Further, the stitching 16 helps to provide a tight, compressive fit to the wearable sleeve aid 102. The stitching 16 also serves to secure the wearable sleeve aid 102 in place when worn on the body. The stitching 16 may serve, in one or more non-limiting embodiments, to define the pocket area 104, whereby the size and shape of the pocket area 104 corresponds to the targeted recovery area of the body. In one or more non-limiting embodiments, the stitching 16 may extend closer to the boundaries of the pocket area 104 where a removable pack, such as removable pack 12, can be inserted and removed from the pocket area 104 of the wearable sleeve aid 102. In one or more non-limiting embodiment, the stitching 16 may be triple stitching. In other words, multiple stitches are used to securely stitch the first inner protective layer 10 and the second outer protective layer 14 together so that the first inner protective layer 10 and the second outer protective layer 14 are tightly bound together. It is noted that in alternative embodiments, the first inner protective layer 10 and the second outer protective layer 14 may be coupled via adhesive material instead of or in addition to the stitching 16. Notably, any means of attaching the first inner protective layer 10 to the second outer protective layer 14 may be used as known in the art without limitation.

In one or more non-limiting embodiments, wearable sleeve aid 102 includes a removable pack 12. As shown in FIG. 1, the removable pack 12 is identified by broken lines that track or match the outer boundary of the pocket area 104 of the wearable sleeve aid 102. In other word, the pocket area 104 defines a cavity within which the removable pack 12 is designed to fit. Accordingly, the removable pack 12 is configured to be inserted and removed into and out of the pocket area 104 formed between the first inner protective layer 10 and second outer protective layer 14, whereby the removable pack 12 is configured to fit a size and shape of the pocket area 104. In one or more non-limiting embodiments, the removable pack 12 may be a gel pack that may be heated or cooled separately. Alternatively, the removable pack 12 may include any other material other than or in addition to gel.

The removable pack 12 is configured to retain heat upon heating the removable pack 12 and to trap cold upon cooling the removable pack 12 in order for the removable pack 12 to achieve a desired temperature for a period of time. For example, a user may heat the removable pack 12 with a heating device, such as a microwave, radiator, oven, or any other useful heating device. To cool the removable pack 12, the user may place the removable pack 12 in a refrigerator or freezer or any other cooling device to cool down the removable pack 12 to the desired cold temperature.

Advantageously, the first inner protective layer 10 functions as a protective layer to prevent discomfort to a wearer of the wearable sleeve aid 102 when the removable pack 12 is inserted into the pocket area 104 of the wearable sleeve aid. By having the first inner protective layer 10, the user is able to avoid having a cold or hot removable pack 12 directly contact or touch his or her skin and/or the targeted recovery area on the user's body, because direct contact with a hot or cold removable pack 12 may be uncomfortable to the user. Rather, the first inner protective layer 10 protects the targeted recovery area of the user's body from an uncomfortable direct contact with the removable pack 12. Many conventional cooling or heating packs require the user to provide some sort of barrier or protective layer on their own before applying the conventional cool or heating packs to one's skin or other targeted recovery area on the user's body, which is why people often have to wrap up a conventional pack in a towel, a plastic outer shell, or some other item. Advantageously, the wearable sleeve aid 102 includes the first inner protective layer 10 built into the wearable sleeve aid 102 so that the user can apply the wearable sleeve aid 102 without any additional inconvenience or difficulty, and is also configured to be worn on the user's body and the targeted recovery area. The user is able to slide the wearable sleeve aid 102 over the targeted recovery area such that the first inner protective layer 10 touches the user's skin and targeted recovery area of the user's body.

In one or more non-limiting embodiments, the removable pack 12 may be made of or may include ELASTOGEL. One of ordinary skill in the art will appreciate that any other type of material may be used or included in the removable pack 12, including non-gel like materials. The removable pack 12 can be cooled or heated and helps reduce swelling or pain for an injured area of the user's body or an area that needs to be relaxed and rehabilitated. The removable pack 12 preferable is flexible and does not leak or dry out. In one embodiment, the removable pack 12 is configured to both be cool or hot when an external source is used to bring to a desired temperature. In other embodiments, the removable pack 12 may solely be cooled or solely be heated.

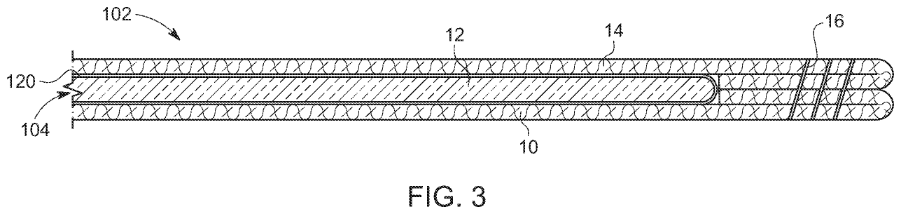

To insert or remove the removable pack 12 (including when the removable pack 12 has been pre-heated or cooled to a desired temperature), the pocket area 104 may include an opening 120 (e.g. as shown in FIG. 3) located on either a top side or a bottom side of the second outer protective layer 14 that provides access to the pocket area 104. The opening 120 of the pocket area 104 may have a means of accessing the pocket area 104 which may include, but is not limited to, having a foldable flap, a zipper, a fastener device (not shown), or a combination thereof covering the opening 120 to further secure the removable pack 12 in place when located within the pocket area 104. The opening 120 to the pocket area 104 may be positioned along the top, bottom, sides, or middle area of the pocket area 104. The opening 120 may be a wide or narrow opening 120 without limitation.

In one or more non-limiting embodiments, instead of the pocket area 104 being accessible from the second outer protective layer 14, the opening 120 may be located on either a top side or a bottom side or other part of the first inner protective layer 10 thereby providing access to the pocket area 104. Further, the opening 120 may be secured by a foldable flap, a zipper, a fastener device, or a combination thereof, without limitation. That being said, in other embodiments, there may not be anything covering the opening 120 to the pocket area 104 such that the pocket area 104 is exposed. Either way, it is intended for the user to be able to slide in or otherwise place the removable pack 12 into and out of the pocket area 104 of the wearable sleeve aid 102.

In one or more non-limiting embodiments, an external securing device 18 is attached to one or more layers of the wearable sleeve aid 102. In one non-limiting embodiment, the external securing device may be attached to at least the second outer protective layer 14 of the wearable sleeve aid 102, whereby the external securing device 18 is configured to provide additional compression as well as to secure the removable pack 12 in place when the removable pack 12 is positioned within the pocket area 104. In some non-limiting embodiments, the external securing device 18 may be attached to both the first inner protective layer 10 and the second outer protective layer 14 or solely to the first inner protective layer 10 and extend over the pocket area 104 of the wearable sleeve aid 102.

In one or more non-limiting embodiments, the external securing device 18 includes one or more straps that can be wrapped and tightened over the second outer protective layer 14, and thus also over the pocket area 104 and the removable pack 12 located within the pocket area 104. In one or more non-limiting embodiments, the external securing device 18 may include one or more straps that can be secured in place using hook and loop pieces, i.e. VELCRO, so that the user may easily attach and detach the external securing device 18 over the second outer protective layer 14 and the underlying pocket area 104 on the wearable sleeve aid 102. Alternatively, or additionally, the external securing device 18 may comprise one or more straps that may be loosed or tightened over the pocket area 104 and may include fasteners (e.g. buttons or snaps) or zippers, or a combination of fasteners, zippers, and/or hook and loop pieces without limitation thereto.

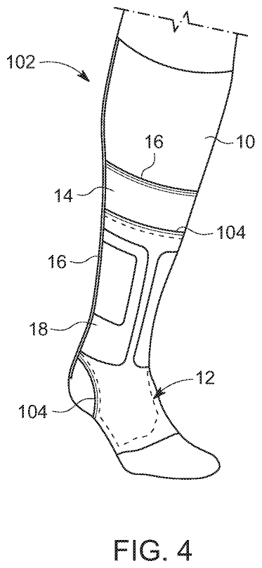

Alternatively, instead of one or more straps, the external securing device 18 may be in the form of one or more zippers, fasteners, or hook and loop pieces that can be secured around the pocket area 104 and the inserted removable pack 12 so as to provide additional compression over the targeted recovery area of the user's body. Regardless of whether the external securing device 18 includes one or more straps having the VELCRO pieces (e.g. as shown in FIG. 1 and FIG. 4), zippers, or fasteners, or whether the external securing device comprises a zipper, fastener, or VELCRO pieces that can be secured over the pocket area 104, the external securing device 18 provides a means of additional compression. In particular, when the user has secured the external securing device 18 over the pocket area 104 of the wearable sleeve 102, the external securing device 18 provides additional compression over the removable pack 12 when positioned within the pocket area 104. The pocket area 104 is intended to align with the targeted recovery area of the user's body, and thus, the compression provided by the external securing device 18 also aligns with and targets the targeted recovery area of the user's body. The external securing device 18 may further function to secure the wearable sleeve aid 102 in place such that the wearable sleeve aid 102 does not move around.

It is noted that the second outer protective layer 14 can further function as another source of static compression. This may be because of the use of the stitching 16 that couples the first inner protective layer 10 to the second outer protective layer 14 which secures the first inner protective layer 10 and the second outer protective layer 14 together and also forms a pocket area 104 in between the two layers 10, 14. This may also be due to the type of material used for the inner layer 10 and the outer layer 14. In one or more non-limiting embodiments, the first inner protective layer 10 and the second outer protective layer 14 may be made of the same type of material. Alternatively, the second outer protective layer 14 may be made of a different material than the first inner protective layer 10. In one or more non-limiting embodiment, the first inner protective layer 10 and the second outer protective layer 14 may be made using soft and breathable material, but also a material that provides a tighter fit. An example of such a material may be DRI-FIT material. However, those of ordinary skill in the art will understand that any other type of material may be used for the first inner protective layer 10 and second outer protective layer 14 of the wearable sleeve aid 102, including without limitation, cotton, rayon, polyester, spandex, or a combination thereof. Further, in a non-limiting embodiment, the external securing device 18 may include a combination of neoprene as well as the VELCRO (i.e. hook and loop pieces.)

Advantageously, the wearable sleeve aid 102 may help the user fulfill the recommended steps of the RICE method. The letters in the RICE method stand for “Rest, Ice, Compression, and Elevation.” Accordingly, the user is able to wear the wearable sleeve aid 102 and achieve the tenets of the RICE method, because the user is able to apply cold or heat to the injured area of the user's body using the removable pack 12. Further, the wearable sleeve aid 102 includes many built in means of compression to provide compression to the injured area of the user's body due to the combination of an external securing device 18. As noted above, the external securing device 18 provides additional compression when secured in place over the second outer protective layer 14 and the underlying pocket area 104.

Further, as noted above, the second outer protective layer 14 functions as a compression type layer due to the tight fit between the second outer protective layer 14 and the first inner protective layer 10. The incorporation of compression in the wearable sleeve aid 102 (as per the RICE method) is very beneficial. Compressing one's injury helps reduce and prevent swelling. Swelling typically causes more pain to an injury since the swelling puts a lot of pressure on the injured area. Since compressing can reduce swelling, the compression ultimately helps with lessening a user's pain as well. Beneficially, the wearable sleeve aid 102 shown in FIGS. 1-6 provides a means for the user to use a removable pack 102 that can be either cooled or heated which provides pain relief and therapeutic benefits to a targeted part of a user's body, which may also be an injured area. Further, the wearable sleeve aid 102 provides means for compression (e.g. via the external securing device 18 and/or the second outer protective layer 14) which also provides pain relief and therapeutic benefits to the targeted recovery area.

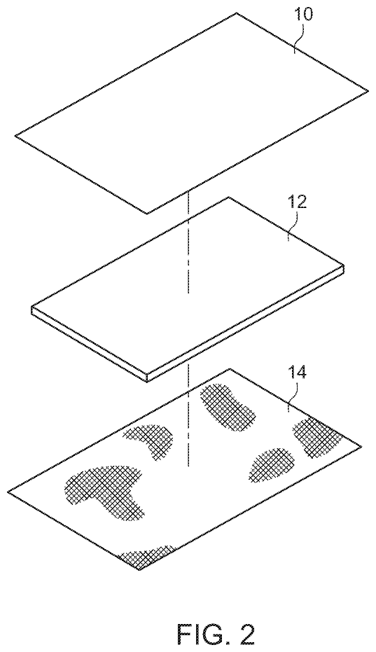

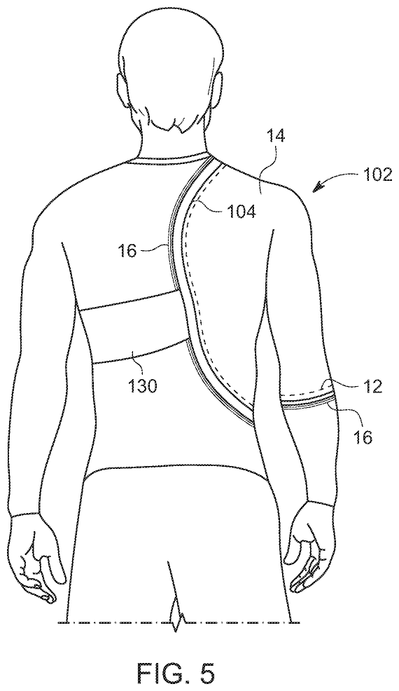

Turning to FIG. 2, FIG. 2 is an exploded view in accordance with a non-limiting embodiment. FIG. 2 shows the multiple layers that form the wearable sleeve aid 102 in one or more non-limiting embodiment. As shown in FIG. 2, the wearable sleeve aid 102 may include a first inner protective layer 10 coupled together with a second outer protective layer 14. The removable pack 12 is intended to be positioned between the first inner protective layer 10 and the second outer protective layer 14. As noted above, stitching 16, as shown in FIG. 1, FIG. 3, FIG. 4, and FIG. 5, may be used to attach the first inner protective layer 10 to the second outer protective layer 14 to one another, whereby a pocket area 104 (e.g. as shown in FIG. 1, FIG. 3, FIG. 4, and FIG. 5) is formed between the first inner protective layer 10 and the second outer protective layer 14. The removable pack 12 is positioned within the pocket area 104 between the first inner protective layer 10 and second outer protective layer 14.

Referring to FIG. 3, FIG. 3 provides a sectional view taken along line 3-3 in FIG. 1 in accordance with a non-limiting embodiment. As shown in FIG. 3, the wearable sleeve aid 102 includes the first inner protective layer 10 and the second outer protective layer 14. Stitching 16 may be used to attach the first inner protective layer 10 to the second outer protective layer 14, whereby a pocket area 104 is formed between the first inner protective layer 10 and the second outer protective layer 14. The removable pack 12 is configured to be inserted into the pocket area 104 formed between the first inner protective layer 10 and second outer protective layer 14. Further, the wearable sleeve aid 102 may include an opening 120 located on either a top side or a bottom side of either the first inner protective layer 10 or the second outer protective layer 14 (e.g. as shown in FIG. 3) that provides access to the pocket area 104. While not shown in FIG. 3, the opening 120 may have a foldable flap, a zipper, or a fastener device (not shown) covering the opening 120 to further secure the removable pack 12 in place when located within the pocket area 104.

FIG. 4 is a perspective view in accordance with a non-limiting embodiment. FIG. 4 shows the wearable sleeve aid 102 configured to slide over a user's foot and lower part of the leg. In one or more non-limiting embodiments, the wearable sleeve aid 102 functions as a sock or a device that can be slid over the foot and lower part of a user's leg. The wearable sleeve aid 102 of FIG. 4 incorporates the first inner protective layer 10 and the second outer protective layer 14. A form of stitching 16 is used to attach the first inner protective layer 10 to the second outer protective layer 14, whereby the pocket area 104 (as discussed above) is formed between the first inner protective layer 10 and the second outer protective layer 14. The stitching 16 serves to define the pocket area 104 and one or more boundaries of the wearable sleeve aid 102, whereby the size and shape of the pocket area corresponds to the targeted recovery area of the body. The stitching 16 also serves to secure the wearable sleeve aid 102 in place when worn on the body. As shown in FIG. 4, the first inner protective layer 10 of the wearable sleeve aid 102 may extend beyond the pocket area 104 and portion of the wearable sleeve 102 incorporating the second outer protective layer 14. The wearable sleeve aid 102 of FIG. 1 and FIG. 5 may also have a portion of the first inner protective layer 10 that extends beyond the stitching 16 and/or the second outer protective layer 14 in one or more non-limiting embodiments.

It is noted that the wearable sleeve aid 102 can target a direct part of a user's body (e.g. the ankle or the shin or calves as shown in FIG. 4), but also functions to rehabilitate, relax, and/or alleviate any pain or tension in the supporting muscles and elements of a user's body that surround a specific or targeted part of the user's body. For example, in FIG. 1, the wearable sleeve aid 102 allows a user to target the elbow, but also provides support via the compression and securing of the wearable sleeve aid 102 of the forearm and biceps of the user's body. Similarly, in FIG. 4, the wearable sleeve aid 102 provides rehabilitation and therapeutic benefits to the user's ankle and shins, but also provides support to any other elements in the lower leg of the user. Thus, the surrounding muscles are also benefited from the wearable sleeve aid 102.

In one or more non-limiting embodiments, a removable pack 12 is configured to be inserted into the pocket area 104 shown in FIG. 4 formed between the first inner protective layer 10 and second outer protective layer 14, whereby the removable pack 12 is configured to fit a size and shape of the pocket area 104. The removable pack 12 is represented by the broken lines that trace or follow the boundaries of the pocket area 14. Accordingly, the removable pack 12 may be customized to fit the pocket area 104. Further, the location of the pocket area 104 is site specific or injury specific depending on the body part that the wearable sleeve aid 102 is to be applied to. In one or more non-limiting embodiments, an external securing device 18 is attached to at least the second outer protective layer 14, whereby the external securing device 18 is configured to provide additional compression as well as to secure the removable pack 12 in place when the removable pack 12 is positioned within the pocket area 104.

FIG. 5 is a perspective view in accordance with a non-limiting embodiment. FIG. 5 shows the wearable sleeve aid 102 configured to slide over the shoulder of the body. In one or more non-limiting embodiments, the wearable sleeve aid 102 fits over the shoulder and arm and back of the user. The wearable sleeve aid 102 includes a portion that slides over the user's arm and another portion that covers the user's shoulder and back. The wearable sleeve aid 102 may further include one or more straps or wraps 130 that help the user position wearable sleeve aid 102 over the user's shoulder and back. Any of the wearable sleeve aids 102 may include additional straps, such as straps or wraps 130, that help secure the wearable sleeve aid 102 in place. The straps 130 may be separate from or may be in conjunction with an external securing device 18 that can be secured over a pocket area 14 of the wearable sleeve aid 102 in one or more non-limiting embodiments. FIG. 5 does not show the external securing device 18 located over the pocket area 14 of the wearable sleeve aid 102. Nevertheless, it is intended that the wearable sleeve aid 102 shown in FIG. 5 may include an external securing device 18 in accordance with the description above for the external securing device 18 of FIG. 1 and FIG. 4. The wearable sleeve aid 102 shown in FIG. 5 includes a removable pack 12 that may be positioned within the pocket area 104 formed between the first inner protective layer 10 and the second outer protective layer 14.

As shown in FIG. 5, if a user has an injury to his or her shoulder or his or her back, the wearable sleeve aid 102 may be large enough to provide benefits to either the shoulder or back, and may also support any surrounding supportive elements or muscles of the user's targeted recovery area. In other non-limiting embodiments, the wearable sleeve aid 102 may be specific to a narrow segment or specific body part and may not necessarily include enough additional material to cover all of the surrounding supportive muscles and elements surrounding a specific body part where an injury may have occurred.

The wearable sleeve aid 102 may be used for any part of a user's body without limitation. Accordingly, the wearable sleeve aid 102 may be designed for specific parts of a user's body, including, but not limited to, the head, neck, shoulders, arms (including elbows), back, abdomen, groin, hips, legs, feet, hands, or any other part of the body, including smaller parts or elements of each of the body parts listed above. The wearable sleeve aid 102 is injury specific or site specific to a specific part of the user's body such that the user may purchase or obtain a wearable sleeve aid 102 for a particular part of the user's body. Further, the pocket area 104 in the wearable sleeve aid 102 for any specific body part needing recovery (i.e. the targeted recovery area) is located in the proper placement to best ensure that the removable pack 12 can pass along its heating or cooling properties to the targeted recovery area. Further, the wearable sleeve aid 102 may come in a variety of sizes, such as, but not limited to small, medium, large, and x-large, to cover the different sized bodies and body parts.

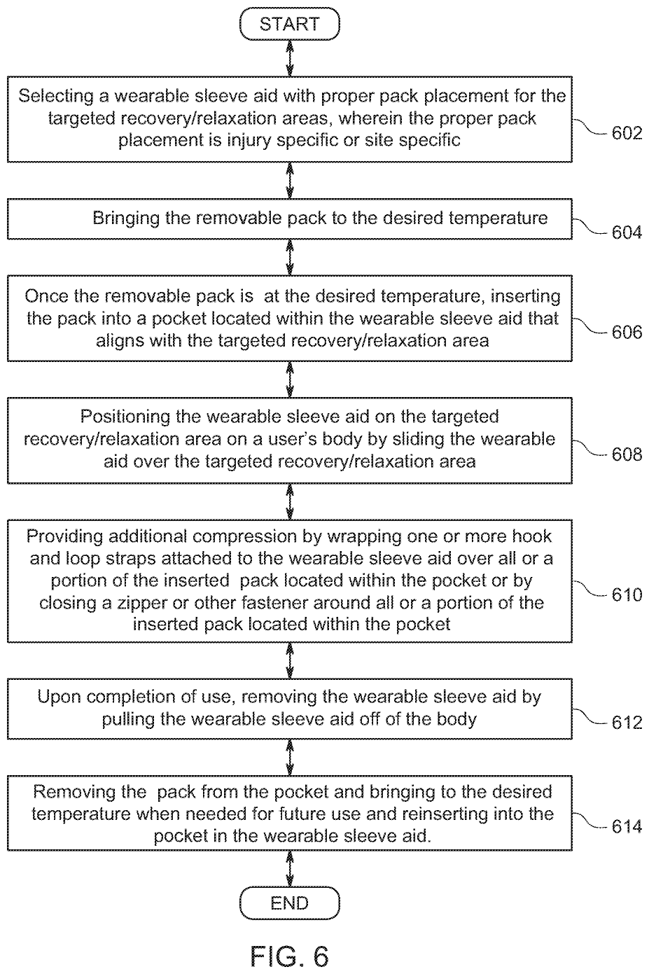

Turning to FIG. 6, FIG. 6 is a flowchart of an exemplary method for using a wearable sleeve aid 102 described above. In one or more non-limiting embodiments, the process may begin at step 602 by selecting a wearable sleeve aid 102 having a removable pack 12 and having proper pack placement for a targeted recovery area of a user's body, wherein the proper pack placement is injury specific or site specific. As noted above, if a user needs to apply the removable pack 12 to his or her shoulder, the user may select a wearable sleeve aid 102 intended to be slid over and cover all or part of the user's shoulder and having the proper pack placement (i.e. pocket area 104) to cover the injury. As further noted above, in some embodiments, the wearable sleeve aid 102 may also include additional material to target surrounding muscles of the user's body such as his or her arm or back (e.g. as shown in FIG. 5).

At step 604, the method may continue by bringing the removable pack 12 to a desired temperature. Continuing to step 606, upon reaching the desired temperature, the removable pack 12 may be inserted into a pocket area 104 located within the wearable sleeve aid 102.

At step 608, the process may continue with positioning the wearable sleeve aid 102 on the targeted recovery area on the user's body by sliding the wearable sleeve aid 102 over the targeted recovery area. As noted above, the selected wearable sleeve aid 102 may be a size that best fits the user and fits over the targeted area of the user's body. Accordingly, the wearable sleeve aid 102 has an opening on either end of the top and bottom of the wearable sleeve aid 102 that allows the user to slide the wearable sleeve aid 102 over and onto a targeted recovery area of a user's body.

At step 610, the process may continue by providing additional compression to the targeted recovery area by securing an external securing device 18 in place over the pocket area 104 and over the second outer protective layer 14 of the wearable sleeve 12. As noted above, in some non-limiting embodiments, the external securing device 18 may be secured by wrapping one or more straps over the pocket area 104 of the wearable sleeve aid 102, whereby the one or more straps include one or more hook and loop pieces, zippers, and/or fasteners. Alternatively, or additionally, the external securing device 18 may not necessarily include one or more wraps or straps, but rather may comprise one or more zippers, fasteners, hook and loop pieces that by themselves surround the pocket area 107 on the second outer protective layer 14 and can be secured in place over the pocket area 104 to provide additional compression as desired.

At step 612, upon completion of use, the process may include removing the wearable sleeve aid 102 by pulling the wearable sleeve aid 102 off of the user's body. The process may continue at step 614 by removing the removable pack 12 from the pocket area 104. The user may store the removable pack 12 either in a separate location (e.g. in a refrigerator or freezer or medicine cabinet). Alternatively, the user may keep the removable pack 12 stored in the wearable sleeve aid 102. When needed for future use, the user may bring the removable pack 12 to the desired temperature and reinsert the removable pack 12 into the pocket area 104 in the wearable sleeve aid 102.

Advantageously, the wearable sleeve aid 102 provides the user with a therapeutic, rehabilitative aid that a user can wear anywhere at any time as a brace or support with or without the removable pack 12 being positioned within the pocket area 104 of the wearable sleeve aid 102 or cooling or heating the removable pack 12. The wearable sleeve aid 102 may be worn over or under the user's clothes, or directly on the user's body without another barrier or layer of clothing in between.

The wearable sleeve aid 102 further provides the user with flexibility and mobility such that the user can easily carry a removable pack 12 within the wearable sleeve aid 102 that offers rehabilitative and relaxation benefits directly on a targeted area of the users' body while located within the wearable sleeve aid 102. Thus, unlike a majority of existing therapeutic heating or cooling devices available on the market, the user is not stuck to one place (e.g. a bed or chair) while wearing the wearable sleeve aid 102 because the user is either unable to move or the user is afraid that the wearable sleeve aid 102 will fall off of the user's body because the aid is not secured properly. There are multiple was in which the wearable sleeve aid 102 is designed to stay secured in place. As noted above, the wearable sleeve aid 102 slides on and may have a tight fit depending on the size selected by the user. Further, the first inner protective layer 10 and the second outer protective layer 14 may be stitched together tightly with additional stitching positioned around the borders of the wearable sleeve aid 102 (e.g. as shown in FIG. 1 and FIG. 4) to help keep the wearable sleeve aid 102 in place. Further, an external securing device 18 is attached to the wearable sleeve aid 102 to further provided compression and secure the wearable sleeve aid 102 in place.

Another benefit of the wearable sleeve aid 102, as noted above, is that the wearable sleeve aid 102 provides a protective first inner protective layer 10 to protect the user from direct contact with a very hot or cold removable pack 12.

Many other advantages and benefits are offered by the wearable sleeve aid as described herein in one or more non-limiting embodiments. The wearable sleeve aid 102 as described and shown in the accompanying Figures may provide a useful and unique means for providing rehabilitative and relaxation aid for a targeted area of the body.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand the invention for various embodiments with various modifications as are suited to the particular use contemplated. The present invention according to one or more embodiments described in the present description may be practiced with modification and alteration within the spirit and scope of the appended claims. Thus, the description is to be regarded as illustrative instead of restrictive of the present invention.

What is claimed is:

1. A wearable sleeve aid configured to function as a rehabilitative and relaxation aid for a targeted recovery area of a body, the wearable sleeve aid comprising:
a first inner protective layer;
a second outer protective layer;
a pocket area, wherein the pocket area is located between the first inner protective layer and the second outer protective layer;
a removable pack, wherein the removable pack is configured to be inserted into the pocket area formed between the first inner protective layer and second outer protective layer; and
an external securing device attached to either the first inner protective layer or the second outer protective layer or both the first inner protective layer and the second outer protective layer, wherein the external securing device is configured to provide additional compression as well as to secure the removable pack in place when the removable pack is positioned within the pocket area.

2. The wearable sleeve aid of claim 1, wherein the removable pack is configured to retain heat upon heating the removable pack and to trap cold upon cooling the removable pack in order for the removable pack to achieve a desired temperature.

3. The wearable sleeve aid of claim 1, wherein the first inner protective layer functions as a protective layer to prevent discomfort to a wearer of the wearable sleeve aid when the removable pack is inserted into the pocket area of the wearable sleeve aid.

4. The wearable sleeve aid of claim 1, wherein the second outer protective layer is configured to provide a source of static compression.

5. The wearable sleeve aid of claim 1, wherein the first inner protective layer and the second outer protective layer are stitched together, wherein the pocket area is formed between the first inner protective layer and the second outer protective layer and is defined by the stitching.

6. The wearable sleeve aid of claim 1, further comprising additional stitching that runs along the boundaries of the wearable sleeve aid to further secure the wearable sleeve aid in place when worn on the body.

7. The wearable sleeve aid of claim 1, wherein the wearable sleeve aid further comprises an opening located on either a top side or a bottom side of the first inner protective layer that provides access to the pocket area, the opening having a foldable flap, a zipper, or a fastener device covering the opening to further secure the removable pack in place when located within the pocket area.

8. The wearable sleeve aid of claim 1, wherein the wearable sleeve aid further comprises an opening located on either a top side or a bottom side of the second outer protective layer providing access to the pocket area, wherein the opening is secured by a foldable flap, a zipper, or a fastener device.

9. The wearable sleeve aid of claim 1, wherein the external securing device comprises one or more straps that can be tightened and loosened, wherein the one or more straps are configured to provide additional compression over the removable pack when positioned within the pocket area and further secures the wearable sleeve aid over the targeted recovery area of the body.

10. The wearable sleeve aid of claim 1, wherein the external securing device comprises one or more straps that are securable using hook and loop pieces, zippers, or one or more fasteners.

11. The wearable sleeve aid of claim 1, wherein the wearable sleeve aid is configured to be slid over an arm or an elbow of a body.

12. The wearable sleeve aid of claim 1, wherein the wearable sleeve aid is designed to be slid over a foot, a leg, or a shoulder of the body.

13. The wearable sleeve aid of claim 1, wherein the removable pack is configured to fit a size and shape of the pocket area.

14. The wearable sleeve aid of claim 13, wherein the size and shape of the pocket area corresponds to and aligns with the targeted recovery area of the body.

15. A method of using a wearable sleeve aid, the method comprising:
selecting a wearable sleeve aid having a removable pack and having proper pack placement for a targeted recovery area of a user's body, wherein the proper pack placement is injury specific or site specific, wherein the wearable sleeve aid comprises a first inner protective layer coupled to a second outer protective layer, wherein a pocket area is defined by the first inner protective layer and the second outer protective layer;
bringing the removable pack to a desired temperature;
upon reaching the desired temperature, inserting the removable pack into the pocket area located within the wearable sleeve aid;
positioning the wearable sleeve aid on the targeted recovery area on the user's body by sliding the wearable sleeve aid over the targeted recovery area;
providing additional compression to the targeted recovery area by securing an external securing device coupled to the first inner protective layer or the second outer protective layer of the wearable sleeve aid or secured to both the first inner protective layer or the second outer protective layer, wherein the external securing device is located above the pocket area of the wearable sleeve aid; and
upon completion of use, removing the wearable sleeve aid by pulling the wearable sleeve aid from the user's body

16. The method of claim 15, wherein a position of the pocket area for the wearable sleeve aid aligns with the targeted recovery area.

17. The method of claim 15, further comprising, securing the external securing device in place to provide additional compression by securing one or more straps in place, wherein the one or more straps comprise one or more hook and loop pieces, one or more zippers, or one or more fasteners.

18. The method of claim 15, further comprising, protecting the targeted recovery area of the user's body from direct contact with the removable pack, wherein the first protective inner layer of the wearable sleeve aid protects the targeted recovery area from direct contact with the removable pack and from the desired temperature of the removable pack.

19. The method of claim 15, further comprising, providing additional compression to the targeted recovery area, wherein the second outer protective layer functions to provide additional compression over the pocket area and over the targeted recovery area.

20. The method of claim 15, further comprising, heating or cooling the removable pack by placing the removable pack in a heating device or a cooling device.
